(19) United States
(12) Patent Application Publication
(10) Pub. No.: US 2004/0177101 A1
(43) Pub. Date: Sep. 9, 2004

(54) Electronic infant information recorder

(76) Inventor: Carl Underwood, Costa Mesa, CA (US)

Correspondence Address: Robert D. Fish, 611 Anton Blvd., Suite 1400, Costa Mesa, CA, 92626, US

(21) Appl. No.: 10/382,817

(22) Filed: Mar. 5, 2003

(51) Int. Cl. G06F 17/60; G06F 1/00; A61B 5/00

(52) U.S. Cl. 708/131; 705/2; 600/300

(57) ABSTRACT

The present invention provides a hand-held processing devices having a special function input used to input baby feeding data. The hand-held processing device needs no programming by the user to accept baby feeding data. The phrase “special function input” as used herein, means a control pre-programmed (i.e., programmed at some time before sale to the end user) to cause the hand-held processing device to execute a set of instructions to specifically accept user input comprising baby-related data. The phrase “baby-related data” means any information regarding a parameter measured for a baby that may be recorded, including feeding data, diaper data, sleeping data, temperament data, temperature data, medicine data, and water or other non-milk fluid data, emergency contact data, allergy data, and schedule or appointment data.

Contemplated special function inputs include inputs for diaper data, sleeping data, temperament data, temperature data, medicine data, and water or other non-milk fluid data. The special function input may be physical keys, as well as other input controls, including voice recognition, a touch pad, or a touch pad receiver.

Also, methods of marketing a hand-held device, comprising advertising the device as one specifically providing for input of baby-related information including baby feeding data are contemplated.

### FIELD OF THE INVENTION

The field of the invention is handheld computers.

### BACKGROUND OF THE INVENTION

Various types of computer devices are currently available, some of which are specifically made or marketed for specialized uses.

Monitoring medication for patients is an example of a specialized use. U.S. Pat. No. 6,421,650 issued to Goetz et al. teaches a medication monitoring system and apparatus that comprises three components: (1) a patient component having a retrievable patient database of patient medical history, prior prescribed medications, and data transfer interface; (2) a physician component having a retrievable physician database of medication information and input/output device enabling a prescribing physician to enter prescription information into that component; and (3) a pharmacist component resident on a pharmacist's computer adapted to transfer prescription data from the patient's computer component.

Other specialized uses include monitoring various events or parameters for newborns or infants, such as feeding time, feeding amount, sleeping time, dirty or wet diapers, the baby's environment, etc. Some of these monitors are described below.

WO 02/062282 teaches an infant care unit containing non-contact sensing and monitoring of physiological parameters. The baby is placed within an incubator, while the sensors and lenses monitor physiological parameters. The baby care unit also contains an environment control means that is responsive to the sensor output, and which is capable of changing the environment. This baby monitor does not actually record and store data.

Another type of monitor for infants is mentioned in a conference paper (Krawciw, Natalia et al., Bioengineering, Proceedings of the Northeast Conference, Published by IEEE, IEEE Service Center, Piscataway, N.J. USA 1989), which describes a micro-processor based system designed to evaluate the effects of environmental stimuli on infants in neonatal ICU. That unit receives and records information from various monitoring instruments. It also evaluates various physiological parameters such as heart rate, respiration, blood pressure. Additionally, that unit comprises a handheld computer that is implemented to record an baby's behavioral state. However, that unit may not be easy to use in a residential setting. Since that unit is designed to automatically obtain and record data from various monitoring instruments, the unit may not be user-friendly, thus making it difficult to be used in a home. Additionally, that type of unit is likely very expensive.

Furthermore, personal digital assistants “PDAs” or other handheld computers are available that have the ability to accept, store, and display various types of data. Some of those devices can be programmed to accept and store special types of data or perform particular functions. However, none of those handheld computers are configured to conveniently store baby-related data using special function buttons, keys, and so forth.

Thus, there is still a need for a low-cost, portable computer that quickly stores and retrieves information or parameters, in a user-friendly manner.

### SUMMARY OF THE INVENTION

The present invention provides hand-held processing devices having a special function input used to input baby feeding data. The hand-held processing device needs no programming by the user to accept baby feeding data. The phrase “special function input” as used herein, means a control pre-programmed (i.e., programmed at some time before sale to the end user) to cause the hand-held processing device to execute a set of instructions to specifically accept user input comprising baby-related data. In contrast, the phrase “general function input” as used herein means any control that activates a set of instructions, but is not specifically configured to accept user input for baby-related data.

For example, a PDA user could click on an icon to start Excel, and then create an Excel spreadsheet to store baby input feeding data. At the start of that process, clicking on the Excel icon is using the icon as a general function input. Once the spreadsheet is created, the user could create a desktop shortcut to that spreadsheet, and thereafter that shortcut could be used to access the baby input feeding data. But that would still not be a pre-programmed “special function input”, because it was not programmed at some time before sale to the user. On the other hand, the device would be considered to be pre-programmed if all the user had to do to engage the special function inputs were to type a few codes, or in some other manner activate the device. Thus, selling a general purpose device would not infringe, but selling software that adapted a general function device to provide baby-related special function inputs would comprise contributory infringement.

The term “baby” as used herein means any newborn, infant, toddler, or child whose information or data would be helpful to record. Furthermore, as used herein, the phrase “baby-related data” means any information regarding a parameter measured for a baby that may be recorded, including feeding data, diaper data, sleeping data, temperament data, temperature data, medicine data, and water or other non-milk fluid data. Contemplated special function inputs include inputs for diaper data, sleeping data, temperament data, temperature data, medicine data, and water or other non-milk fluid data.

The special function input is not limited to physical keys. Alternatives include voice recognition, a touch pad, or a touch pad receiver.

The hand-held processing device may advantageously comprise a display that presents choices such as feeding amount or other baby-related functions. The device may also have keys such as up and down arrow buttons, numbered buttons, or buttons containing letters of the alphabet.

Additionally, the present invention provides a method of marketing a hand-held device, comprising advertising the device as one specifically providing for input of baby-related information including baby feeding data.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWINGS

FIG. 1 is a perspective view of a hand-held computer embodying the invention.

FIG. 2 is a top view of the hand-held computer of FIG. 1, having a special function input comprising physical keys.

FIG. 3 is a top view of the hand-held computer of FIG. 1, having a special function input comprising touch pad buttons 240.

FIG. 4 is a top view of the hand-held computer of FIG. 1, having a special function input comprising a touch pad receiver.

FIG. 5 is a top view of the hand-held computer of FIG. 1, having a special function input comprising a voice receiver.

FIG. 6 is a top view of the hand-held computer of FIG. 1, having a

### DETAILED DESCRIPTION

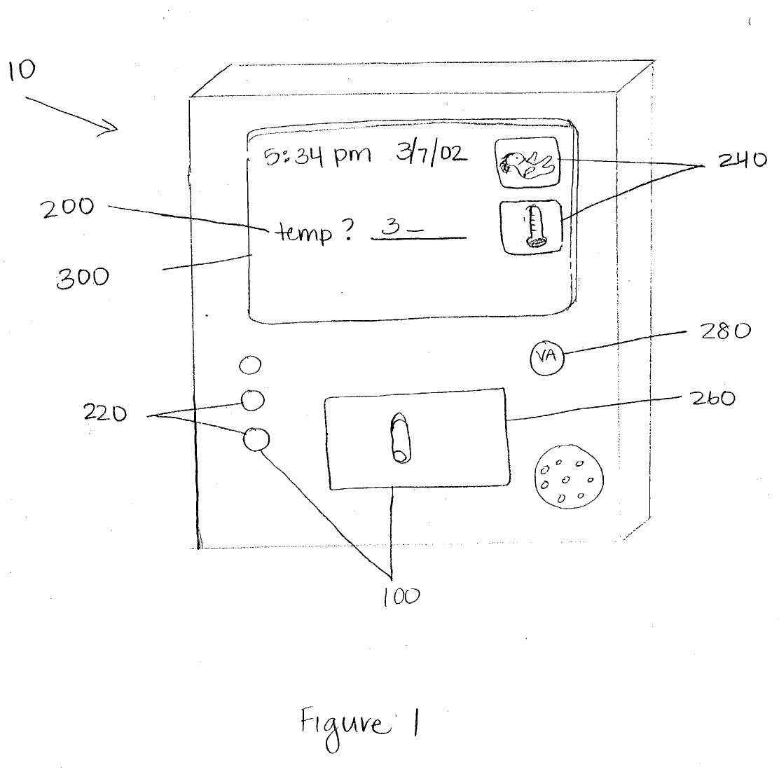

In FIG. 1, a handheld processing device 10 has a special function input 100 used to input baby-related data 200, and a display 300. The special function input 100 may comprise physical keys 220, touch pad buttons 240, a touch pad receiver 260 for receiving a drawn symbol, or a voice activation device 280, so long as at least one special function input 100 is specific for input of baby feeding data.

The handheld processing device is sized and dimensioned to fit easily within an average adult's hand. The handheld processing device is lightweight and portable, thus making it easy to use and store.

The processing device comprises any standard microprocessor used in portable devices that is capable of receiving, storing, and retrieving information. For example, the processing device may be similar to those used in common personal digital assistants (PDAs), or even a multi-function calculator. It is also contemplated that the processing device may process, categorize, and sort the inputted information in some manner. The term “process” as used herein means categorize, file, store, sort, or calculate. For example, the information may be processed and sorted into a chart, which can later be displayed. Or perhaps the information may be categorized or classified according to key words, so that the information may be easily retrieved using any of those categories or classifications.

The subject matter disclosed herein comprises a mechanism of informing the hand-held device that the user wants to input baby-related data so that the hand-held device can prompt the user for that information. Basically, the special function input 100, when selected, allows the user to input baby-related data 200 or other baby-related information. For example, it is contemplated that the special function input 100 may be a shortcut-type button or icon that can be selected by a user, which when selected, will display a window or input box that specifically receives baby-related data 200 or information.

Some examples of special function inputs 100 include physical keys 220, a touch pad receiver 260, touch pad buttons, or voice activation 280, all of which cause the hand-held device 100 to execute a set of instructions to accept input of baby-related data. The handheld device 10 must comprise at least one special function input 100 that is used to input baby feeding data. The term “feeding data” is herein used mean amount of food consumed, and may also include type of food, time food was consumed, or any other useful feeding-related information for babies.

It is not necessary that a pre-programmed special function input be dedicated entirely to the special function. Nor is it necessary that a pre-programmed special function input always retain the special function.

It is preferable that the handheld device 10 has more than one special function input 100, wherein each special function input 100 is used to input specific data. For example, data indicating a wet or dirty diaper may be entered using two separate special function inputs. Also, data regarding sleeping patterns of a baby, or amount of time a baby has slept may be entered using a third special function key or other input. A child's temperament or disposition, temperature at a given time, medication taken or prescribed, and amounts and times of feeding water or other non-milk fluids may also be input using still other special function inputs.

In other embodiments, the device 10 may have an internal clock so that a timer will begin when a special function input 100 is pressed, and stop when that special function input is pressed again. For example, a special function input 100 for sleep time may be pressed to start a timer when the baby goes to sleep, and pressed again to stop the timer when the baby wakes up. The device 10 may also be able to convert the “start” and “stop” times to an amount of time that the baby slept and store that information. However, the device may also store the information in a chart showing “start” and “stop” times for the baby's sleep.

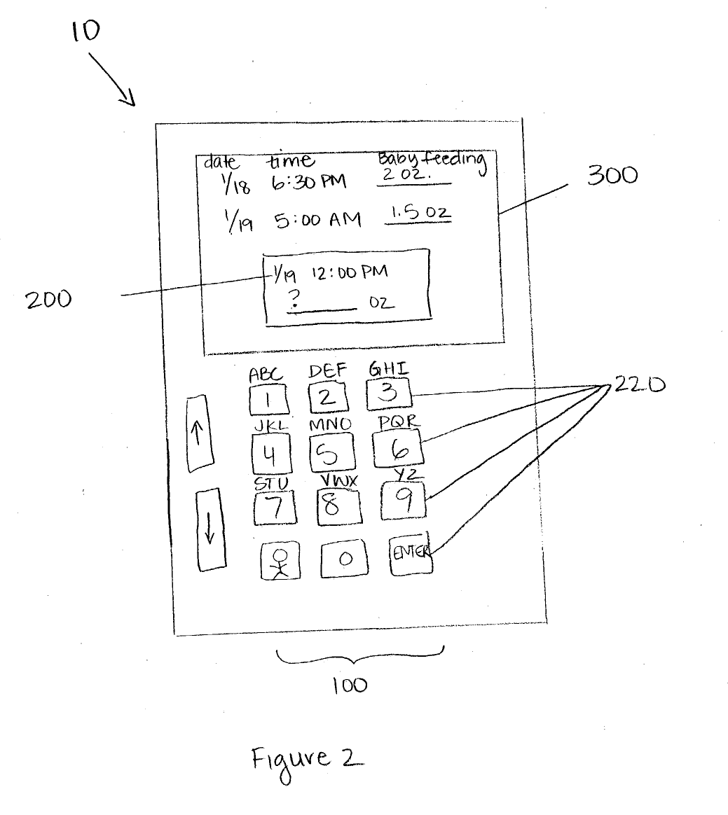

In FIG. 2, the special function input 100 comprises a physical key 220 used to input baby feeding time and amount. It is contemplated that the physical key 220 may be any standard key or button, such as those found on a common keyboard of a computer, or a standard remote control for a TV. The physical key 220 may be identified or labeled with some indication of a baby-related function by, for example, a symbol, icon, word, or any other type of identifying mark. In preferred embodiments, a word or icon such as a baby picture or symbol, is placed on the key, but it is also contemplated that a mark may be placed next to the key to indicate its function.

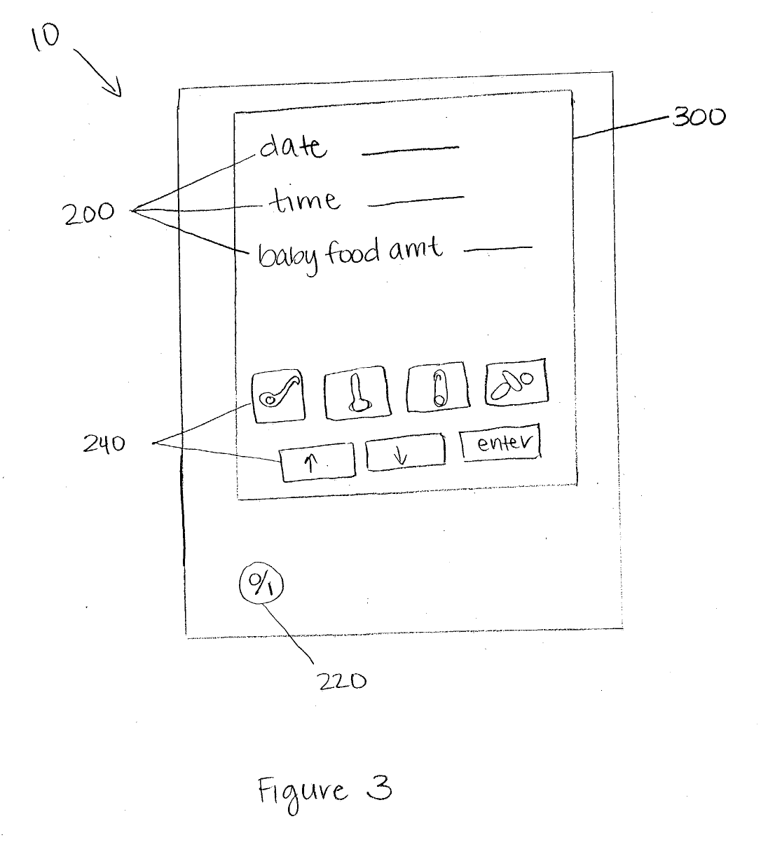

In FIG. 3, the special function input 100 comprises touch pad buttons 240. It is contemplated that the touch pad buttons 240 may be tapped by a finger, stylus 270, or other tool. The touch pad buttons are commonly located on a screen, and may be positioned anywhere on the handheld device 10. Contemplated touch pad buttons 240 may be similar to those found on ATMs, wherein a finger is used to tap the selected button, or on PDAs, wherein a stylus 270 is used to tap the desired touch pad button. The touch pad button(s) 240 may be identified or labeled with some indication of a baby-related function by, for example, a symbol, icon, word, or any other type of identifying mark.

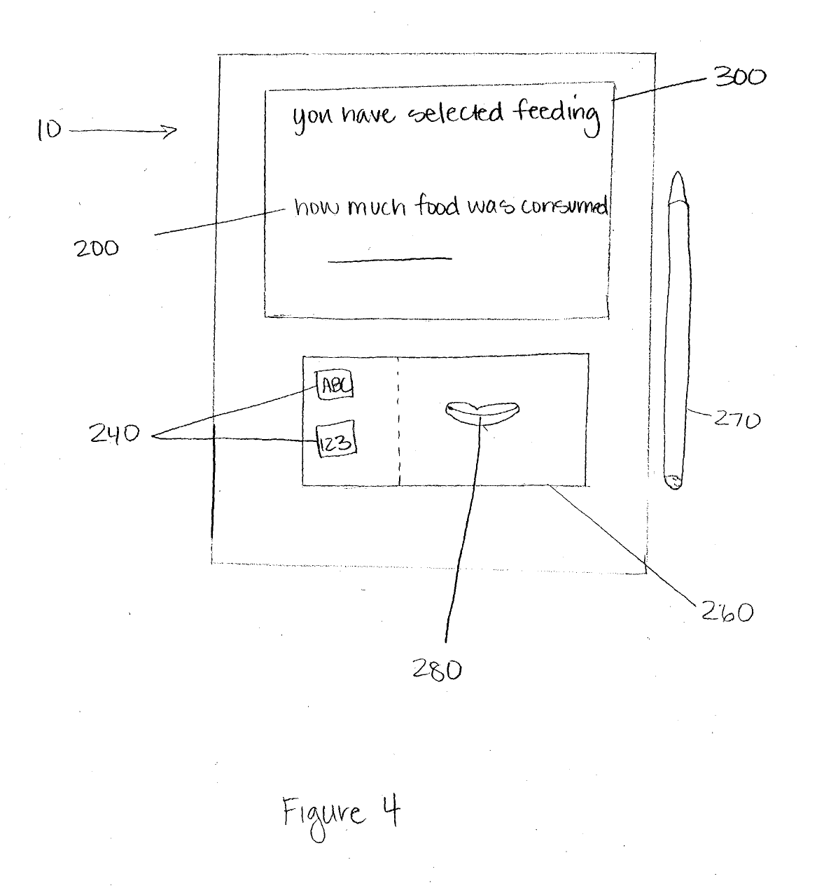

The special function input 100 may further comprise a touch pad receiver 260, as depicted in FIG. 4, which is designed to receive a symbol 280 drawn by a stylus 270 or other similar tool. Similar to the other types of special function inputs 100 mentioned above, the touch pad receiver 260 may be identified or labeled with some indication of a baby-related function by, for example, a symbol, icon, word, or any other type of identifying mark. The touch pad receiver 260 may comprise any touch pad receiver, similar to those found on PDAs. Another example of a contemplated type of touch pad receiver 260 is a signature receiver that is often used to store electronic signatures during a credit card transaction. Thus, the user may draw an icon or other symbol using a pen or stylus 270 on the touch pad receiver 260, which would activate software or other set of instructions to accept user input, such as baby feeding data or other baby-related data 200.

Still another contemplated special function input 100 may be voice activation hardware and software 280, as shown in FIG. 1. Thus, it is contemplated that the handheld device 10 has a microphone or other recording device 10 designed to work in conjunction with software to recognize and record data. In preferred embodiments, a control is selected to activate the voice recognition software.

It is contemplated that the handheld computer may have any number of layout designs or styles of special function input(s) to capture a large market and to enable various users to be able to easily and quickly use the device 10. It is contemplated that there are several ways a user can select a special function input 100. For example, a separate key may be configured to allow one specific type of baby-related information to be input. As another example, one key may be configured to allow various types of baby-related information to be input. In that type of configuration, after the one key is selected, a window may pop-up showing different baby-related input categories. The baby-related input category can be selected by using a general function control, such as a shift control, arrow controls, or an ENTER control.

Another contemplated design includes a physical menu button for each type of baby-related data 200 that may be input into that handheld device 10. In that type of design, the user can select the control that will receive the type of data that the user would like to input or enter. Other contemplated styles or designs include having an alpha-numeric keypad labeled 0-9, similar to that on a telephone, or a keyboard similar to those on computers, wherein each key can be used to input numbers or letters, or some combination of both. Such a keyboard or telephone keypad type system are examples of general function inputs. The alpha-numeric keys may also have special function input 100 capabilities to allow for input of specific baby-related information. In that type of design, it is contemplated that a general function input such as a SHIFT or FUNCTION control (which might, for example, be labeled BABY, or have an image of a baby) may be used in conjunction with an alpha-numeric key to bring up a window or input box, allowing the user to input specific baby-related information. For example, if the SHIFT control and the key labeled “6” are simultaneously depressed, a window might be displayed requesting information regarding the amount of food the baby ate. The user would then be able to use the numbered keys to input the number of ounces of food the baby ate. Thus, it may be possible for one key or control to have general function input and special function input capabilities.

In a preferred embodiment, the special function input 100 comprises one physical key 220 and various general function controls, such as up and down arrows, which will allow the one physical key 220 to record different types of data. Thus, when the one physical key 220 is selected, perhaps a window will display different categories of input data and the user can use the up and down arrows to select one category. In another embodiment, the physical key 220 can have more than one function. That type of design allows the handheld computer to be carried easily as well as user-friendly.

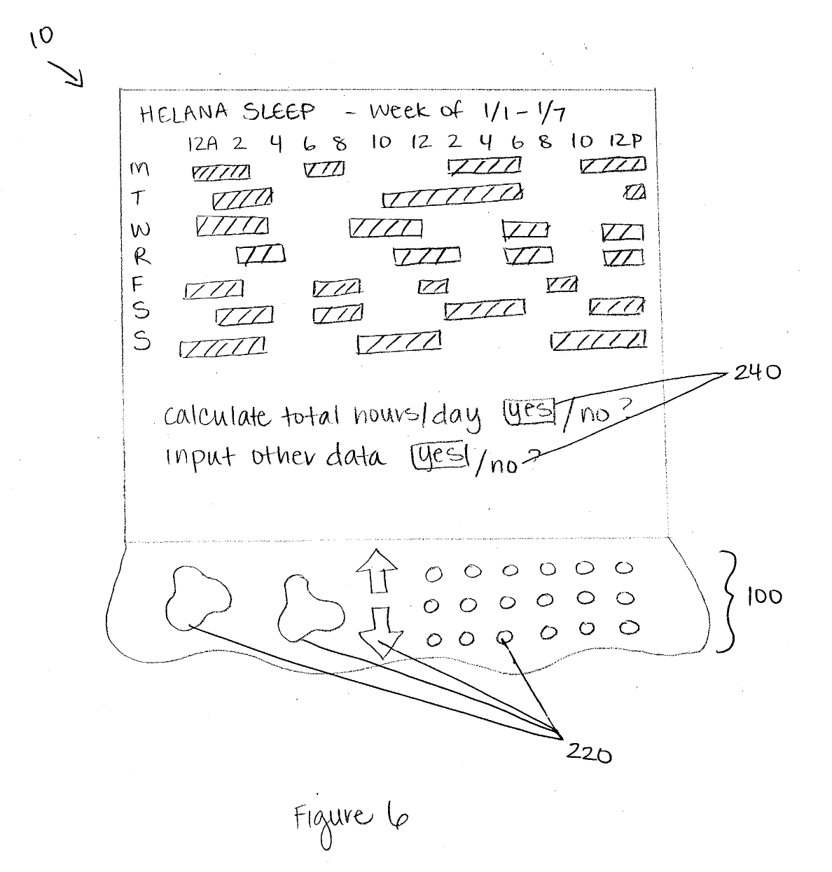

The handheld computer further comprises a display 300. The display 300 may vary in style and layout, to accommodate and facilitate the needs of various users. For example, the display 300 may present data that was entered in the previous entry, data for the entire day, data for a week (depicted in FIG. 6), etc. Additionally, the display 300 may present choices for feeding amount, or any other information that may be input, or may just provide a blank screen or space for data to be entered (see FIG. 6). Thus, it is contemplated that the display 300 may be more free-style, allowing the user to input various data, or it may be more structured, wherein the display 300 may suggest entries, or may store and provide a pop-up of the last entry, or may ask questions and allow the user to simply fill in the blank.

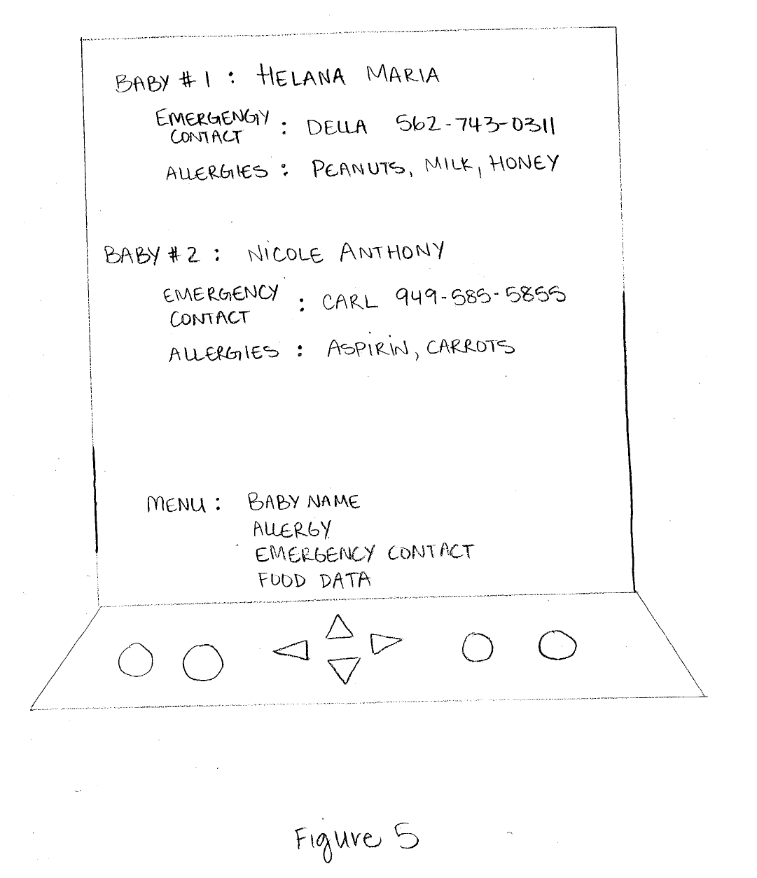

In preferred embodiments, the display 300 presents choices for at least one of the following baby functions: at 1 oz of food, ate 2 oz of food, ate 3 oz of food, refused to eat, wet diaper, dirty diaper, clean diaper, 1 hour of sleep, 2 hours of sleep, slept through the night, cranky temperament, and happy temperament. In further preferred embodiments, the device 10 may be capable of storing emergency contact information of a parent or other guardian, as well as relevant baby information, including allergies or habits, or medicines being given to the baby, as depicted in FIG. 5. The device 10 may also be used to record and store doctor appointments for the baby, and other types of appointments and schedules. In other preferred embodiments, the device 10 automatically records and displays a current time and date (see FIG. 1), thereby simplifying the task of inputting data by the user. For example, when the user tries to input data, the display 300 may suggest a current date and time, providing “yes” or “no” choice buttons for the user (See FIG. 6).

Users may find it helpful to give the device to a baby-sitter or other caretaker so that they can easily access important information on the baby. For example, a baby-sitter would be able to easily access emergency information, allergies, types of medication that the baby may be taking, etc. It is also contemplated that the device 10 may be capable of storing information for more than one baby (depicted in FIG. 5).

In still other contemplated embodiments, the hand-held processing device 10 may be co-branded to include the brand Disney®, Pooh®, Toys-R-Us®, Gerber®, WB®, etc. Additionally, the handheld computer may display advertisements for various products. It is contemplated that the handheld computer may display prerecorded advertisements, or may receive and display advertisements through a cellular or other signal. In other contemplated embodiments, the computer may display recommendations, based on the data being presently input. For example, if a user is currently inputting data that the baby's temperature is too high, the display 300 may recommend or suggest that the baby be given a specific medicine, or may provide suggestions for how to reduce the fever (i.e. place the baby in a luke-warn water bath).

Still other embodiments include triggering or activating an alarm as a reminder to perform some activity. For example, a user may be able to set an alarm to remind the user of a baby's doctor appointment, or when to give the baby medicine, etc. Additionally, it is contemplated that the device 10 may record important milestones in a baby's life. For example, a user could input the date the baby first started walking, the date the baby lost his or her first tooth, etc.

Regarding methods, the present invention provides a method of marketing a hand-held device, comprising advertising the device as one specifically providing for input of baby-related information including baby feeding data. In contemplated embodiments, the hand-held device is a hand-held microprocessor having a pre-programmed special function input used to record or input baby feeding data, or other baby-related data. In an especially preferred embodiment, the hand-held device is a PDA having (at least) four special function inputs (four physical buttons) used to input each of: (1) amount of food consumed; (2) amount of non-milk liquid consumed; (3) diaper data (i.e. wet, dirty, clean); and (4) temperament of baby. Furthermore, the marketing may comprise advertising, brochures or other written literature, TV or radio commercials, word of mouth, slogans, and other sorts of advertising or marketing materials.

Thus, specific embodiments and applications of handheld computers have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A hand-held processing device having a pre-programmed special function input used to input baby feeding data.

2. The hand-held processing device of claim 1 wherein the special function input comprises a physical key.

3. The hand-held processing device of claim 2 wherein the physical key contains a word or symbol on or next to the key.

4. The hand-held processing device of claim 1 wherein the special function input comprises a button on a touchpad.

5. The hand-held processing device of claim 1 wherein the special function input comprises a symbol that may be tapped by a stylus.

6. The hand-held processing device of claim 1 wherein the special function input comprises a special character that may be drawn using a stylus.

7. The hand-held processing device of claim 1 wherein the device further comprises a special input used to input an amount of water or other non-milk fluid consumed by a baby.

8. The hand-held processing device of claim 1 wherein the device further comprises a special input used to input at least one of the following data: diaper data, sleeping data, or temperament data, emergency contact data, allergy data.

9. The hand-held processing device of claim 1 wherein the device further comprises a special input used to input at least one of the following data: temperature data or medicine data, or appointment data.

10. The hand-held processing device of claim 1 wherein the device further comprises a display that presents data for an entire day.

11. The hand-held processing device of claim 1 wherein the device further comprises a display that presents choices for feeding amount.

12. The hand-held processing device of claim 1 wherein the device further comprises a display that presents choices for at least one of the following baby functions: wet diaper, dirty diaper, 1 hour of sleep, 2 hours of sleep, cranky temperament, and happy temperament.

13. The hand-held processing device of claim 1 wherein the device further comprises a special input used to input at least one of the following data: diaper data, sleeping data, or temperament data.

14. The hand-held processing device of claim 1 wherein the device automatically records a current date and time.

15. The hand-held processing device of claim 1 wherein the device is co-branded.

16. The hand-held processing device of claim 1 wherein the device displays advertisements.

17. The hand-held processing device of claim 1 wherein the device displays recommendations.

18. A method of marketing a hand-held device, comprising advertising the device as one specifically providing for input of baby-related information including baby feeding data.

19. The method of claim 18 wherein the device further comprises a pre-programmed special function input used to input baby feeding data.

20. The method of claim 19 wherein the special function input comprises a physical key for inputting baby feeding data.
